(19) United States
(12) Patent Application Publication
(10) Pub. No.: US 2007/0154966 A1
(43) Pub. Date: Jul. 5, 2007

(54) Assay for the detection of rapamycin and rapamycin analogs

(76) Inventor: Ronit Sagi-Eisenberg, Ness-Ziona (IL)

Correspondence Address: NATH & ASSOCIATES, 112 South West Street, Alexandria, VA, 22314, US

(21) Appl. No.: 10/584,770

(22) PCT Filed: Dec. 29, 2004
(86) PCT No.: PCT/IL04/01172

§ 371 Date: Jun. 28, 2006

Related U.S. Application Data
(60) Provisional application No. 60/532,552, filed on Dec. 29, 2003.

(51) Int. Cl. G01N 33/53

(52) U.S. Cl. 435/7.92

(57) ABSTRACT

The present application discloses an assay and a kit for quantification of rapamycin in various samples.

### FIELD OF THE INVENTION

The present invention concerns a method for the detection of rapamycin and rapamycin analogs, as well as a kit for use in said method.

### BACKGROUND OF THE INVENTION

Rapamycin (sirolimus), a macrocyclic lactone, generated by Streptomyces hygroscopicus, was initially identified 20 years ago during antibiotic screening and found to display remarkable anti fungi activity. Subsequently, rapamycin was recognized to possess highly potent immunosuppression properties and has since been used as the drug of choice in organ transplantation¹. More recently, the growth inhibitory effects of rapamycin have been recognized alongside the elucidation of the molecular basis of its function². The ultimate cellular target of rapamycin has been identified as a signaling kinase named “mTOR” that plays a central role in the control of cell proliferation and survival.

It became clear that through rapamycin binding to its intracellular receptor protein FKBP12, a complex is formed that inhibits the function of mTOR³. This inhibition then results in cell cycle arrest in the G1 phase. Currently, mTOR is the only identified target of rapamycin, which places this drug in a unique position of being the most selective kinase inhibitor known. Based on these intriguing findings, the potential clinical applications of rapamycin have gone much beyond its initial development as an immunosuppressant. Rapamycin-coated cardiac arterial stents are already in clinical use to prevent intima overgrowth⁴ and the evaluation of rapamycin and rapamycin analogs as anti cancer drugs has begun⁵⁻⁸. Indeed, in phase I/II clinical trials rapamycin and its analogs have already demonstrated impressive growth-inhibitory effects against a broad range of human cancers.

Pharmacokinetic and clinical data show that rapamycin is a critical-dose drug requiring at least monthly monitoring of drug concentrations in the blood^{1,9}. However, current assays are based on high performance liquid chromatography (HPLC) coupled to mass spectrometry (MS). These methodologies require special equipment and skills and are accordingly conducted only in specialized laboratories around the world.

As it appears that the use of rapamycin is going to increase dramatically in the near future, it would clearly be advantageous to have available a simple assay for monitoring blood levels of rapamycin and rapamycin analogs. An assay that would not require any special equipment or skills and that could be performed on a routine basis in any hospital laboratory.

### SUMMARY OF THE INVENTION

The method of the present invention, contrary to standard monitoring techniques for rapamycin and rapamycin analogs, such as HPLC and MS, provides a simple, cheap and easily conducted assay for the determination of this important drug in various samples.

The present invention concerns an assay for determining rapamycin or rapamycin analog concentrations in a sample comprising:
(i) contacting the sample with PKBP12 protein, or with a rapamycin binding fragment of said PKBP12 protein that maintains the rapamycin binding-properties, for a time period and under conditions allowing formation of rapamycin/FKBP12 complex;
(ii) contacting the rapamycin/FKBP12 complex with a complex-binding domain of mTOR for a time period and under conditions enabling binding of the complex to said complex-binding domain;
(iii) detecting the amounts of said complex-binding domain to the rapamycin/FKBP12 complex;
(iv) comparing the amounts detected in (iii) to a calibration curve, thereby determining the rapamycin concentrations in the sample.

In accordance with the present invention, the term “rapamycin” used hereinafter refers both to the native rapamycin as generated by streptomyces hygroscopicus, as well as to a synthetically produced rapamycin, or to any analog of the two.

The term “sample” refers to a liquid sample, preferably a liquid sample of a body fluid such as plasma, blood, serum, urine, sperm, cerebral spinal fluid, and the like. The term further relates to a solid or semi-solid sample such as tissues, feces, and the like, or, alternatively, to a solid tissue such as those commonly used in histological diagnosis. Preferably, the sample is one which may contain blood or other cells in which rapamycin may be detected.

An especially preferred sample is blood, and most preferably serum. Preferably, the specimen is a mammalian blood or serum sample.

The first step in the assay of the invention is contacting the sample with either the full FKBP12 protein being a 12 kDa protein, or alternatively with a fragment of FKBP12 protein that still maintains the rapamycin binding properties of the full protein. The contact between the sample and the FKBP12 protein or a fragment thereof should be for a sufficient time period and under conditions which enable the formation of a rapamycin/FKBP12 complex.

The time period may be based on knowledge of kinetics of such complexation reactions or on prior experimentation. As known, the kinetics of such reactions depends on a number of factors such as temperature, pH, salt concentration, stoichiometric ratios of the agents undergoing complexation and others. The conditions may be such that allow complexation, per se.

The formation of the complex may be monitored at any stage after the protein or a fragment thereof and the sample were brought into contact with each other, using methods such as various spectroscopic methods, analytical methods or others known to a person skilled in the art. The formation of the complex may be determined based on, for example, aliquots of samples taken out from the reaction vessel during complex formation or spectroscopic determination of complex formation in the vessel without separating aliquots therefrom or combinations thereof. For example, the formation of the complex to be monitored may be calibrated by a method that would provide the analyst with parameters such a spectroscopic point of reference e.g. a wavelength for every stage of the complexation and which would be indicative to such parameters as rate of complex formation or to concentration of complex already formed under the conditions used. The methods of monitoring may be any method known in the art, such as, and without being limited thereto, dipstick assays, FPIA, EMIT, ELISA, VISTA, RIA and MEIA; the determination of the amounts of complex formation, e.g., immobilized complex-binding domain, is preferably achieved by an ELISA reader, in which case a colorimetric reaction provides a reading which is proportional to the amounts of bound complex binding domain.

Preferably, the FKBP12 protein or the fragment thereof is immobilized on a solid substrate such as glass, polystyrene, polypropylene, polyethylene, dextran, nylon, amylases, natural and modified celluloses, polyacrylamides, agaroses or magnetite. These solid substrates may take on various shapes such as single or multi-cuvets, plates, and the like. Preferably the solid substrate is in a form suitable for an ELISA reader. Typically such substrate is in the form of a microtiter plate. Such immobilization enables subsequent detection of the color reaction produced by immobilized compounds.

By a preferred embodiment, the solid substrate (for example microtiter plate) is blocked by non specific protein such as bovine serum albumin (BSA), ovalbumin (OVA) or keyhole limpet hemocyanine (KLH) or any other protein capable of forming a conjugate. The blocking solution is subsequently removed by washing, for example by phosphate buffered saline (PBS), and diluted samples are added. These samples are preferably diluted blood samples. Alongside with the tested samples, known concentrations of rapamycin are added as standards to provide a calibration curve. The concentrations of these controls are typically predetermined by other methods of quantification as known in the art.

After step (i) of the method, a rapamycin/FKBP12 complex is formed. This complex is allowed to bind, under suitable conditions, with a complex binding domain of mTOR. The term “complex binding domain of mTOR” refers to any fragment of mTOR which is known to bind the complex between the internal ligand FKBP12 and rapamycin. Typically, this is a domain having 93 amino acids (called the FRB domain) but may be any other fragment of the kinase that maintains such a complex binding properties.

As typically the FKBP12, or the rapamycin binding fragment thereof, is immobilized onto the solid substrate, said complex-binding domain also becomes immobilized to the substrate upon its binding to the complex. Surplus amounts of the free complex-binding domain may be rinsed. In step (iii) of the method, the amounts of the complex-binding domain of mTOR bound to the rapamycin/FKBP12 complex may be determined, simply by determining the amounts of immobilized complex-binding domain. The methods of determination may be those disclosed hereinabove.

By one embodiment, the complex-binding domain such as the FRB fragment is directly bound to a detectable label, such as a fluorescent material, a chemiluminescent material, a material capable of producing a signal which may be detected by spectrophotometry or gaseometry or an enzyme capable of producing, in the presence of a suitable substrate, a color reaction. For example, in the case the label is an enzyme, the enzyme may be alkaline phosphatase which produces a color reaction in the presence of alkaline phosphate, Horseradish-peroxidase (HRP) enzyme or any other enzyme known in the art capable of giving a colorimetric reaction detectable by optical density (O.D) reading. Each of those enzymes is used with a color-forming reagent or reagents (substrate) such as p-nitrophenyl phosphate or hydrogen peroxide and o-phenylenediamine, respectively. The colorimetric reaction may for example be achieved by a conjugated enzyme which generates the substrate for a second enzyme which gives a colored end-point; the conjugated enzyme converts a pro-enzyme into an enzyme which catalyzes a reaction with a colored compound involved; the conjugated enzyme catalyzes a reaction wherein substrate or end-product can be stained easily.

By another alternative, said complex-binding domain (such as the FRB) may be detected by antibodies which themselves are conjugated to a detectable label or to an enzyme capable of producing a colorimetric reaction in the presence of suitable substrates, such as alkaline phosphatase, HRP enzymes and the like.

The labeled antibodies may be directed against said complex-binding domain (such as the FRB domain), i.e. elicited by immunizing an animal with FRB or an immunogenic fragment thereof. Preferably, and for ease of production, the antibodies may be directed against a tag which is attached to said complex-binding domain. The term “tag” refers herein to an agent selected from a larger number of possible molecules, including but not restricted to peptides, lipids, proteins, nucleotides and others. Examples of peptides are Flag, His, HA, and myc.

A change in color may be monitored by a standard ELISA reader and thus translated into rapamycin concentrations. It is possible to correlate each specific O.D. reading to a corresponding value of a rapamycin concentration based on rapamycin concentration calibration curves produced using rapamycin standards as described above. Alternatively, such calibration curves may for example be produced based on previously obtained results, by performing the method of the invention, under the same conditions, on a plurality of samples with known varying amounts of rapamycin or its analogs.

The present invention further concerns a kit for the determination of rapamycin concentrations, or rapamycin analog concentrations in a sample, the kit comprising:
(i) PKBP12 protein or a rapamycin binding portion thereof immobilized on a solid substrate; and
(ii) a complex binding domain of mTOR linked to a label that can be detected or which can generate a signal.

As indicated above in connection with the method, the FKBP12, or the rapamycin binding portion thereof, may be immobilized on a 96-well microtiter plate. Preferably, the immobilized FKBP12 is blocked with a suitable amount of an unrelated protein such as BSA.

The kit further comprises, optionally in a separate vessel, a complex-binding domain of mTOR preferably the 93-amino acids FRB domain, linked to such an agent which may be detected or that can generate a signal. Preferably this agent is capable of producing a colorimetric reaction.

By one embodiment of the kit, said complex-binding domain is conjugated directly to an enzyme capable of producing, in the presence of a suitable substrate, a colorimetric reaction. Such enzyme may be alkaline phosphatase enzyme, HRP enzyme, and the like.

By another option, the kit of the present invention contains, in an additional separate vessel, antibodies which are themselves conjugated to an enzyme capable of producing a colorimetric reaction, such as alkaline phosphatase or HRP. These antibodies should be conceptually viewed as belonging to element (ii) above as they enable the detection of the said complex-binding domain.

The antibodies may be directed against the FRB fragment directly, but preferably, the FRB is conjugated to a tag of any type, and the antibodies are directed against the tag as explained above in connection with the method.

Optionally, the kit further comprises the substrate required to produce the colorimetric reaction, such as in the case of alkaline phosphatase P-Nitrophenyl phosphate, commercial 3,3′,5,5′-Tetramethylbenzidine (TMB) in the case of HRP and the like.

The kit may also comprise instructions for use and calibration curves to be used in the quantification of rapamycin. Alternatively to providing calibration curves as part of the kit, pre-weighed samples of rapamycin and rapamycin analogs for producing these calibration curves may be provided. This would allow the person using the assay to generate the required curves in real time and under the same set of conditions as the tested sample.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 depicts the results of cloning of FKBP12 and FRB.

FIG. 2 depicts purification of GST-FKBP12: Lane 1—molecular weight ladder; Lane 2—Bacteria before IPTG induction; Lane 3—Bacteria+1 mM IPTG; Lane 4—1^{st} elution; and Lane 5—2^{nd} elution.

FIG. 3 depicts purification of His-FRB: Lane 1—molecular weight ladder; Lane 2—Bacteria before IPTG induction; Lane 3—Bacteria+1 mM IPTG; Lane 4—Effluent; Lane 5—1^{st} elution; Lane 6—2^{nd} elution; Lane 7—3^{rd} elution.

FIG. 4 depicts the results of monitoring rapamycin concentration in accordance with the method of the present invention.

### DETAILED DESCRIPTION OF THE INVENTION

Rapamycin binds specifically and with high affinity to FKBP12, a 12 kDa protein. This complex then binds to a 93 amino acids domain (FRB) present within mTOR, the cellular target of rapamycin. The complex between FKBP12 and FRB is strictly dependent on the presence of rapamycin. The dependency of the complex on rapamycin is used for both the qualitative and quantitative determinations of rapamycin in accordance with the presence invention.

In one specific example, a 96 well microtiter plate is coated with GST-FKBP12 in the range of 50-1000 ng/well. The plate is then blocked by adding a blocking solution such as 2% BSA and/or 2% skim milk to a final volume of 200 μl. The blocking solution is then removed and increasing concentrations of rapamycin (serving as standards for the calibration curve) are added alongside the tested rapamycin containing samples (such as diluted blood samples). Rapamycin is kept as solid and stock solutions prepared and kept for 1 month at 4° C.

By one option, after incubation, a recombinant protein comprising FRB fused to a reporter enzyme such as alkaline phosphatase (AP) is added. By a second option, FRB is produced as an epitope tagged protein by cloning FRB cDNA, produced for example, but not only, by PCR, into an appropriate tag carrying vector (tag options include: Flag, His, HA, myc as mentioned hereinbefore). By a third option, FRB is directly labeled by a detectable material. Preferably, after the addition of tagged FRB to the plate, antibodies, which are directed against the tag and conjugated to a detectable material or to a reporter enzyme such as HRP are added.

Following an incubation period, the wells are washed four times to remove any unbound protein. Under these conditions, only FRB bound to the rapamycin-FKBP12 complex will remain immobilized in the wells, the amounts of the immobilized FRB bound to the rapamycin-FKBP12 complex being proportional to the respective rapamycin concentration.

After washing, the amount of bound antibodies is determined, for example by adding the substrate of the reporter enzyme such as TMB (in the case of HRP). Incubation is then continued until a clear color develops (typically within 20-120 min).

At the end of this incubation period, O.D. is measured by an ELISA reader, for example at 450 nm in the case of HRP/TMB The amount of TMB hydrolyzed reflects the amount of HRP in each well, which in turn is dependent on the amount of FRB retained in each well.

There are many different labels and methods of labeling known to those of ordinary skill in the art. Examples of the types of labels which can be used in the present invention: include enzymes, radioisotopes, fluorescent compounds, colloidal metals, chemiluminescent compounds, phosphorescent compounds, and bioluminescent compounds. Those of ordinary skill in the art will know of other suitable labels for binding to the complexes for example, or will be able to ascertain such, using routine experimentation. Furthermore, the binding of these labels to the complexes formed may be done using standard techniques common to those of ordinary skill in the art.

A kit of the invention may comprise a carrier means being compartmentalized to receive in close confinement one or more container means such as vials, tubes, and the like, each of the container means comprising one of the separate elements to be used in the method. For example, one of the container means may comprise the substrates for producing any of the detectable reactions, e.g. colometric reactions. The kit may also have containers containing pre-weighed samples of rapamycin and rapamycin analogues for producing the calibration curves.

As stated hereinbefore, the kit of the present invention may be used in an “ELISA” format to detect the quantity of rapamycin in sample of choice, e.g. blood, serum, plasma. Thus, the FKBP12 protein or a fragment thereof can be affixed to a solid matrix to form a solid support that comprises a package in the subject diagnostic kit. A reagent is typically affixed to a solid matrix by absorption from an aqueous medium although other modes of affixation applicable to proteins and polypeptides well known to those skilled in the art can be used.

Examples of well-known matrices include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding such proteins, or will be able to ascertain such, using routine experimentation.

The reagent species or labeled specific binding agent of a diagnostic kit described herein can be provided in solution, as a liquid dispersion or as a substantially dry power, e.g., in lyophilized form. Where the indicating means is an enzyme, the enzyme's substrate can also be provided in a separate package of a system. A solid support, such as the before-described microtiter plate and one or more buffers can also be included as separately packaged elements in this diagnostic assay system.

The packaging materials discussed herein in relation to diagnostic systems are those customarily utilized in diagnostic systems. The term “package” or “packaged element” refers to a solid matrix or material, such as glass, plastic (e.g., polyethylene, polypropylene and polycarbonate), paper, foil and the like capable of holding within a diagnostic reagent. Thus, for example, a package can be a bottle, vial, plastic and plastic-foil laminated envelope or the like container used to contain a contemplated diagnostic reagent or it can be a microtiter plate well to which quantities of a contemplated diagnostic reagent have been operatively affixed, i.e., linked so as to be capable of being bound.

Experimental Procedures:

1. Production of the Coating Protein (Protein A):

Cloning of FKBP12: cDNA derived from human lymphoma cell line T Jurkat that was enriched in FKBP12 was used to clone FKBP12 by PCR amplification using the following primers:

Sense:
5′-GCCGGATCCAATGGGAGTGCAGGTGGAAACC-3′
Antisense:
5′-CCAAGAATTCTCATTCCAGTTTTAGAAGCTCC-3′

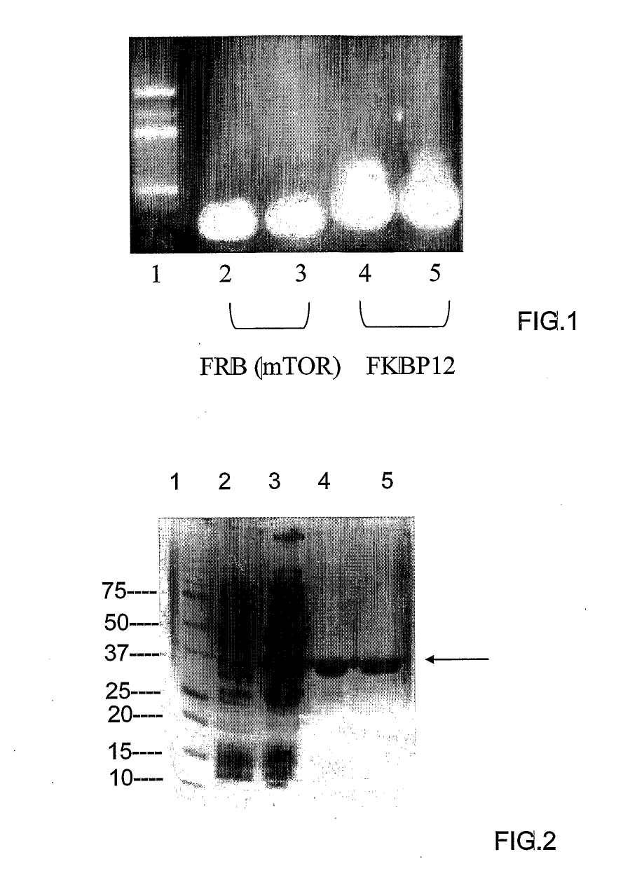

A single PCR product of the expected size of 326 bp was obtained (FIG. 1) and cloned into the EcoRI/BamHI sites of the pGEX vector (Pharmacia). Following transformation into TOP10 cells, the product was confirmed by DNA sequencing.

Production of recombinant GST-FKBP12: Tramsformed TOP10 cells were induced to produce the Fusion protein by 1 mM IPTG at 28° C. for 3 h. GST-FKBP12 was then extracted and purified to homogeneity on Glutathione-Sepharose beads (FIG. 2).

2. Production of Epitope Tagged FRB:

Cloning of FRB: The FKBP12-rapamycin binding domain of mTOR was produced by PCR amplification using the cDNA described above and the following primers:

Sense:
5′-CTAGCTAGCATGTGGCATGAAGGCCTGGAA-3′
Antisense:
5′-CCGCTCGAGAGCTGCTTTGAGATTCGTCGGA-3′

The PCR product obtained (FIG. 1) which corresponds to positions 6155-6423 of mTOR was subcloned in frame into pET-28a vector (Invirogen). Following transformation into C43 cells the product was confirmed by DNA sequencing. This vector was chosen as it provides two possible tags, which could be used (T7 or His). Moreover, the presence of the two His is likely to increase the detection sensitivity.

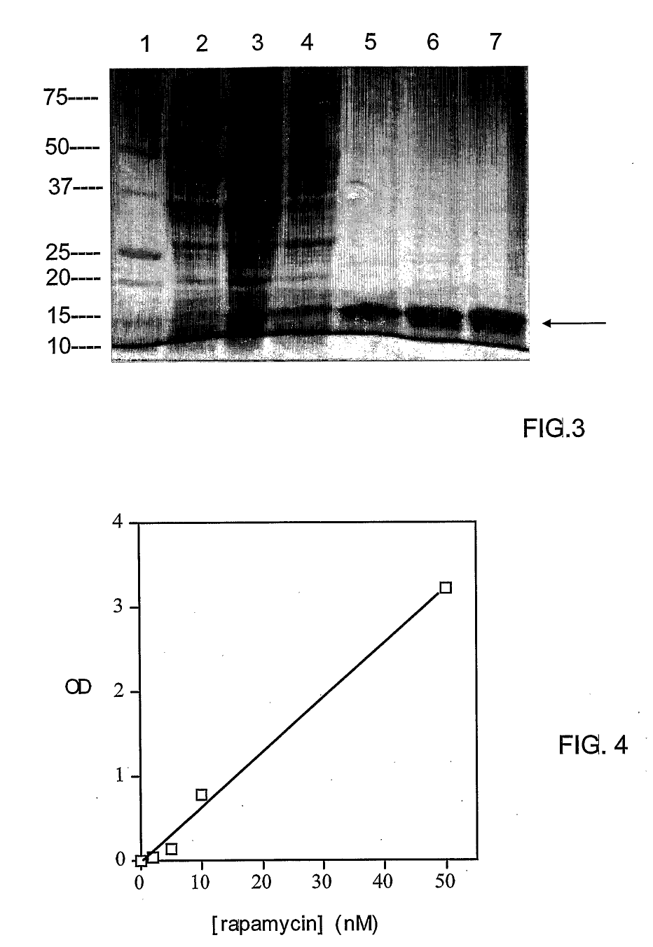

Production of His-FRB-T7-His fusion protein: Transformed C43 cells were induced by 1 mM IPTG at 28° C. for 3 h and the recombinant protein on talon (Clontech) columns (FIG. 3).

3. Monitoring Rapamycin Concentration:

96-well microtiter plates were coated with varying amounts of GST-FKBP12 for 1 h at 4° C. Plates were then washed with TBST and incubated for 2 h with 2% skim milk. After washing, rapamycin and FRB were added at various concentrations and the incubation was continued for 1 h. Plates were then washed 4 times and HRP-conjugated anti His antibodies added at a 1:2500 or 1:5000 dilution. Plates were left over-night at 4° C. and were next washed again 4 times and incubated with 150 μl of a commercial TMB solution for 1 h. Reaction was terminated by the addition of 100 μl of 1M sulfuric acid and the optical density was read in an ELISA reader at 450 nm. Based upon our preliminary experiments, the combination of 50 ng FKBP12, 1.0 pmol FRB and 1:2500 antibody dilution yielded a linear curve which allowed accurate detection of rapamycin at a range of 0-60 nM, which is the pharmacologically relevant range (FIG. 4).

### LIST OF REFERENCES

1) Holt D W Therapeutic drug monitoring of immunosuppressive drugs in kidney transplantation Curr Opin Nephrol Hypertens. 11:657-663.(2002)
2) Keith C T and Schreiber S L. PIK-related kinases: DNA repair, recombination, and cell cycle checkpoints. Science 270: 50-51 (1995).
3) Chen J, Zheng X F, Brown E J and Schreiber S L. Identification of an 11-kDa FKBP12-rapamycin-binding domain within the 289-kDa FKBP12-rapamycin-associated protein and characterization of a critical serine residue. Proc Natl Acad Sci USA. 92:4947-4951 (1995).
4) Cheng-Lai, A, Frishman W H. Sirolimus-eluting coronary stents: novel devices for the management of coronary artery disease. Am J Ther. 11:218-228 (2004).
5) Dancey J E. Clinical development of mammalian target of rapamycin inhibitors. Hematol Oncol Clin North Am. 16:1101-1114 (2002).
6) Huang S and Houghton P J. Inhibitors of mammalian target of rapamycin as novel antitumor agents: from bench to clinic. Curr Opin Investig Drugs. 3:295-304 (2002).
7) Mita M M, Mita A and Rowinsky E K. Mammalian target of rapamycin: a new molecular target for breast cancer. Clin Breast Cancer. 4:126-137 (2003).
8) Guba M, von Breitenbuch P, Steinbauer M, Koehl G, Flegel S, Hornung M, Bruns C J, Zuelke C, Farkas S, Anthuber M, Jauch K W, and Geissler E K. Rapamycin inhibits primary and metastatic tumor growth by antiangiogenesis: involvement of vascular endothelial growth factor. Nat Med. 8:128-135 (2002).
9) Mahalati K and Kahan B D. Clinical pharmacokinetics of sirolimus. Clin Pharmacokinet 40:573-585. (2001)

1. An assay for determining rapamycin or rapamycin analog concentrations in a sample comprising:
(i) contacting the sample with PKBP12 protein, or with a rapamycin binding fragment of said PKBP12 protein that maintains the rapamycin binding properties, for a time period and under conditions allowing formation of rapamycin/FKBP12 complex;
(ii) contacting the rapamycin/FKBP12 complex with a complex-binding domain of mTOR for a time period and under conditions enabling binding of the complex to said complex-binding domain;
(iii) detecting the amounts of said complex-binding domain that is bound to the rapamycin/FKBP12 complex;
(iv) comparing the amounts detected in (iii) to a calibration curve, thereby determining the rapamycin concentrations in the sample.

2. The assay of claim 1, wherein said rapamycin being native rapamycin or synthetically produced rapamycin, or any analog of the two.

3. The assay of claim 1, wherein said sample is a liquid, a solid or a semi solid sample.

4. The assay of claim 3, wherein said liquid sample is a body fluid selected from plasma, blood, serum, urine, sperm, or cerebral spinal fluid.

5. The Assay of claim 3, wherein said solid sample is a tissue.

6. The assay of claim 3, wherein said sample is semi-solid sample selected from tissues or feces.

7. The assay according to claim 4, wherein said liquid sample is mammalian blood.

8. The assay according to claim 1, wherein said FKBP12 protein is full FKBP12 protein being a 12 kDa protein or a fragment of FKBP12 protein that maintains the rapamycin binding properties of the full protein.

9. The assay according to claim 1, wherein the FKBP12 protein or the fragment thereof is immobilized on a solid substrate.

10. The assay according to claim 9, wherein said solid support is a 96-well microtiter plate.

11. The assay according to claim 10, wherein said microtiter plate is blocked by non specific protein.

12. The assay according to claim 1, wherein said detection is achieved by an ELISA reader.

13. The assay of claim 1, wherein said complex binding domain of mTOR is a FRB fragment.

14. The assay according to claim 13, wherein said FRB fragment is directly bound to a detectable label.

15. The assay according to claim 13, wherein said FRB fragment is indirectly bound to a detectable label.

16. The assay according to claim 14, wherein said label is capable of generating a signal detectable by a technique selected from coloremtry, spectrophotometry, fluorospectrophotometry, gaseometry or radiospectrometry.

17. The assay according to claim 16, wherein said detectable label is an enzyme capable of producing, in the presence of a suitable substrate, a color reaction.

18. The assay according to claim 17, wherein said enzyme is alkaline phosphatase or HRP enzyme.

19. The assay according to claim 18, wherein said enzyme is used with a color-forming reagent or reagents selected from p-nitrophenyl phosphate, hydrogen peroxide, o-phenylenediamine and 3,3′,5,5′-Tetramethylbenzidine.

20. A kit for determining rapamycin concentrations, or rapamycin analog concentrations in a sample, the kit comprising:
(i) PKBP12 protein or a rapamycin binding portion thereof immobilized on a solid substrate; and
(ii) a complex-binding domain of mTOR linked to a label that may be detected or that may generate a signal.

21. The kit of claim 20, wherein said complex binding domain of mTOR is provided in separate vessel.

22. The kit according to claim 20, wherein said complex binding domain of mTOR is the 93-amino acids FRB domain, linked to a label that can be detected or that can generate a signal.

23. The kit according to claim 22, wherein said label is capable of generating a signal detectable by a technique selected from coloremtry, spectrophotometry, fluorospectrophotometry, gaseometry or radiospectrometry.

24. The kit according to claim 22, wherein said label is capable of producing a colorimetric reaction.

25. The kit according to claim 22, wherein said label is an enzyme capable of producing, in the presence of a suitable substrate, a colorimetric reaction.

26. The kit according to claim 25, wherein said enzyme is alkaline phosphatase enzyme or HRP enzyme.

27. The kit according to claim 22 further comprising antibodies which are conjugated to an enzyme capable of producing a colorimetric reaction.

28. The kit according to claim 27, wherein said antibodies are directed against the FRB fragment.

29. The kit according to claim 24, wherein said antibodies are directed against a tag to which FRB or FRB fragment are being conjugated.

30. The kit according to claim 22 further comprises the label required to generate a signal detectable by a technique selected from coloremtry, spectrophotometry, fluorospectrophotometry, gaseometry or radiospectrometry.

31. The kit according to claim 22 further comprising pre-weighed samples of rapamycin and rapamycin analogs for producing calibration curves.

32. The assay according to claim 15, wherein said label is capable of generating a signal detectable by a technique selected from coloremtry, spectrophotometry, fluorospectrophotometry, gaseometry or radiospectrometry.
